(19) United States
(12) Patent Application Publication
(10) Pub. No.: US 2021/0336484 A1
(43) Pub. Date: Oct. 28, 2021

(54) RECEIVING UNIT AND POWER TRANSMISSION SYSTEM FOR WIRELESS POWER TRANSMISSION

(71) Applicant: KARDION GMBH, Stuttgart (DE)

(72) Inventors: Samuel Vasconcelos Araujo, Esslingen (DE); Michael Jiptner, Besigheim (DE)

(21) Appl. No.: 17/051,412

(22) PCT Filed: May 2, 2019
(86) PCT No.: PCT/EP2019/061304

§ 371 Date: Jun. 28, 2021

(30) Foreign Application Priority Data
May 2, 2018 (DE) 102018206714.7

(51) Int. Cl. H02J 50/12; A61M 60/178; A61M 60/873

(52) U.S. Cl. CPC H02J 50/12; A61M 60/873; A61M 60/178

(57) ABSTRACT

The invention relates to a receiver unit (200), which is configured to cooperate with a transmitter unit (100) separate from the receiver unit for the wireless transfer of energy, said transmitter unit (100) comprising a primary coil (L₁), which can be supplied with a supply voltage (U_{V}), wherein the receiver unit (200) comprises a secondary coil (L₂), to which a first intermediate circuit capacitor (C_{Z,1}) is connected via a rectifier (210), and a power unit (240), to which a consumer (225) and/or an energy store (220) are connected, wherein the receiver unit (200) comprises a second intermediate circuit capacitor (C_{Z,2}), to which the power unit (240) is connected, wherein the first intermediate circuit capacitor (C_{Z,1}) and the second intermediate circuit capacitor (C_{Z,2}) are connected to one another in a separable manner via a switch (S₇), and wherein the receiver unit (200) comprises an auxiliary supply unit (250), which is connected to the rectifier (210) for the purpose of voltage supply and which is configured to close the switch (S) to connect the first intermediate circuit capacitor (C_{Z,1}) to the second intermediate circuit capacitor (C_{Z,2}) when an output voltage (U_{out,H}) of the auxiliary supply unit (250) exceeds a specified threshold value.

The present invention relates to a receiver unit, which is configured to cooperate with a transmitter unit that is separate from the receiver unit for the wireless transfer of energy, as well as an energy transfer system having such a receiver unit and a transmitter unit for the wireless transfer of energy.

A wireless, in particular inductive, energy transfer can be used for the energy supply of consumers and, in particular, for the charging of energy stores. In this type of energy transfer, a magnetic field can be generated in a transmitter unit with a primary coil; said magnetic field induces a voltage and thus a current flow in a receiver unit with a secondary coil.

This type of energy transfer can be used, in particular, in so-called transcutaneous energy transfer, in which the receiver unit is arranged or implanted under the skin in a human body. Such a transcutaneous energy transfer is advantageous, for example, in circulatory or cardiac support systems (so-called VAD systems, “Ventricular Assist Device”), because there is no permanent wound in the skin through which a cable is guided.

In such an energy transfer system, a power unit can be provided in the receiver unit to be arranged in the body for supplying a consumer or for charging an energy storage unit, said power unit being supplied via the voltage induced in the secondary coil. However, it can be problematic here that the power unit or other parts are disturbed at the beginning of the energy transfer, in particular when, for example, an energy supply unit is not present or is currently disconnected.

Proceeding from the foregoing, the invention is based upon the task of further improving the systems for the wireless transfer of energy known in the prior art and avoiding unstable operating states, in particular during the start-up and power-up phases.

According to the invention, a receiver unit and an energy transfer system having the features of the independent claims are proposed. Advantageous configurations and further developments are the subject matter of the subclaims and the following description.

The invention proceeds from a receiver unit configured to cooperate with a transmitter unit that is separate from the receiver unit for the wireless transfer of energy or from an energy transfer system for the wireless transfer of energy having a transmitter unit and such a receiver unit separate from the transmitter unit. The transmitter unit comprises a primary coil, which can be supplied with a supply voltage. For this purpose, an inverter, for example with suitable semiconductor switches, is generally also provided in order to generate an oscillation of the voltage in the primary coil with a supply voltage present in the form of a DC voltage. An alternating magnetic field can thus be generated by means of the transmitter unit.

The receiver unit correspondingly comprises a secondary coil, to which a first intermediate circuit capacitor, or generally an intermediate circuit capacitance, is connected via a rectifier. The rectifier can be a passive rectifier with suitable diodes, in particular. However, an active rectifier with, for example, suitable semiconductor switches is also advantageous. The first intermediate circuit capacitor, which in the case of energy transfer is charged, is used in particular for smoothing the alternating voltage that is induced in the secondary coil and then rectified. This type of wireless energy transfer is, as already mentioned at the outset, an inductive energy transfer.

In addition, a power unit (or power stage) is provided in the receiver unit, to which power unit a consumer and/or an energy store are connected. While the power unit can be, in particular, a buck converter with, for example, suitable semiconductor switches, an accumulator or a rechargeable battery can be considered as the energy store, in particular.

In a conventional receiver unit, the power stage is generally connected directly to the (single) intermediate circuit capacitor and via the former to the rectifier, so that the power unit is supplied with voltage or energy—via the wireless or inductive energy transfer—when the transmitter unit is active.

However, if the receiver unit does not have an energy store or if the energy store has to be disconnected, for example due to a fault, the receiver unit has to start up independently (in particular, with integrated electronics) as soon as the transmitter unit begins to transfer energy. This means that powering-up or starting-up an auxiliary supply must occur, for example, with signal electronics, control electronics, or the like, which are supplied directly from the intermediate circuit of the resonant converter or via the intermediate circuit capacitor. During this transient start-up phase, an unstable operating state can occur, which can lead to the destruction of the system, i.e. the receiver unit.

According to the invention, it is now provided that the receiver unit has a second intermediate circuit capacitor, to which the power unit is connected, wherein the first intermediate circuit capacitor and the second intermediate circuit capacitor are separably connected to one another via a switch. The intermediate circuit capacitance is thus, so to speak, divided into two parts, which can be connected and separated via the switch. In particular, the first and second intermediate circuit capacitance are connected in parallel when the switch is closed. A semiconductor switch, in particular a MOSFET or IGBT, can be considered as the switch. However, other power circuit breakers are also conceivable.

Further, it is provided that the receiver unit comprises an auxiliary supply unit that is connected to the rectifier for the purpose of supplying voltage and is configured to close the switch in order to connect the first intermediate circuit capacitor to the second intermediate circuit capacitor, i.e. to flip the switch conductively when an output voltage of the auxiliary supply unit exceeds a specified threshold value. This threshold value is preferably 15 V, because all components of the auxiliary supply unit work in their rated voltage range and a stable operating point for the start-up is guaranteed. Thus, it is achieved that the second intermediate circuit capacitor—and thus the power unit—are initially not connected to the secondary coil and the rectifier, but rather, at the start of the energy transfer from the transmitter unit to the receiver unit, only the first intermediate circuit capacitor is initially charged and the auxiliary supply unit is supplied with voltage. The second intermediate circuit capacitor and thus the power unit are connected or switched on only when the output voltage generated by the auxiliary supply unit exceeds the threshold value.

In this way, it can therefore be ensured that no unstable state occurs during start-up, which is usually caused by uncontrolled clocking of power circuit breakers in the power unit, because a relatively high voltage (usually greater than 10 V) is already on the power circuit breaker, while the remaining components such as microcontrollers and driver units have not yet been properly started. With the proposed receiver unit, however, components such as microcontrollers and driver circuits can be fully powered up first. This prevents the unstable state.

The auxiliary supply unit preferably comprises an optical coupler, via which coupler the switch can be actuated. In addition, a voltage comparator can then be provided, which is configured to actuate the optical coupler to close the switch when the output voltage of the auxiliary supply unit exceeds the specified threshold value. In this case, the switch is designed as an N-MOSFET, in particular. The optical coupler can comprise a photodiode and an integrated driver.

Alternatively, it is also preferable for the auxiliary supply unit to comprise a voltage divider for generating the output voltage, said voltage divider being connected to a switching connection of the switch with its voltage divider output. Here, the output voltage is produced, in particular, by dividing the voltage at the first intermediate circuit capacitor. The voltage divider is then connected in such a way, in particular, that the switch is closed when the output voltage exceeds the specified threshold value. In this case, the switch is designed, in particular, as a P-MOSFET, and the voltage divider output is connected to the gate connection of the former, i.e. to its switching connection.

Both variants enable a particularly safe and fast powering-up of the receiver unit and can be used as needed. In both cases, the semiconductor switches used can have a voltage class of 20 V, in particular. In order to reduce losses, values of e.g. 1 mΩ, in particular, may be considered as the drain-source resistance. Likewise, in both cases, a charging of the first intermediate circuit capacitor in case of a connected energy storage unit can be achieved by the intrinsic diode of the MOSFETs, which may be necessary, for example, during regular operation of the receiver unit.

The subject matter of the invention is further an energy transfer system for the wireless transfer of energy having a receiver unit according to the invention and a transmitter unit separate therefrom, which comprises a primary coil that can be supplied with a specified supply voltage.

Although the presented receiver unit and the presented energy transfer system are advantageous for any type of wireless or inductive energy transfer, it is nevertheless particularly expedient for the receiver unit to be configured in such a way that can be arranged, in particular implanted, underneath the skin in a human or animal body, and/or for the transmitter unit to be configured in such a way that it can be arranged on the skin outside of a human or animal body. The energy transfer system (or the receiver unit as a part thereof) thus serves the transcutaneous energy transfer mentioned at the outset.

Further advantages and configurations of the invention can be found in the description and the attached drawing.

The invention is shown schematically in the drawing based upon an exemplary embodiment and is described below with reference to the drawing.

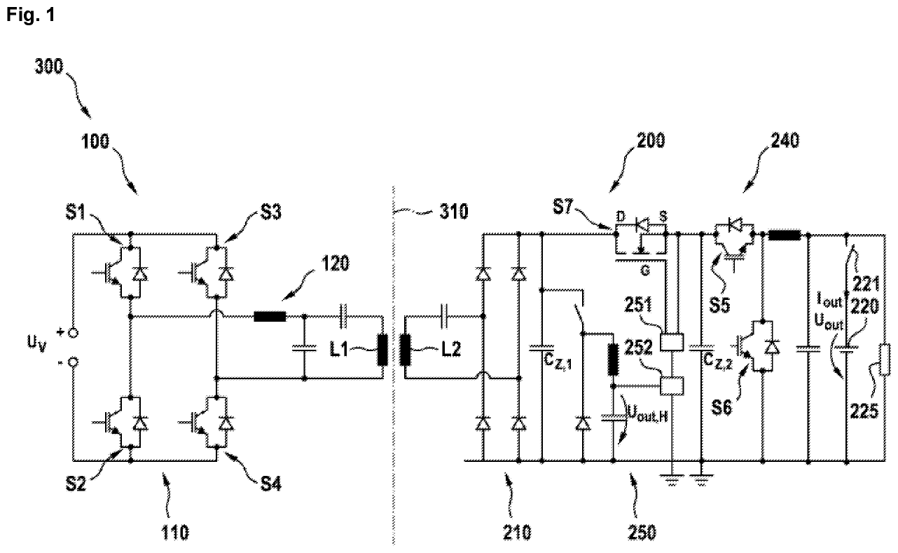

FIG. 1 shows a schematic view of an energy transfer system according to the invention in a preferred embodiment.

FIG. 1 schematically shows an energy transfer system 300 according to the invention for the wireless transfer of energy in a preferred embodiment. The energy transfer system comprises a transmitter unit 100 and a receiver unit 200 separate therefrom, wherein the receiver unit 200 is configured as a receiver unit according to the invention in a preferred embodiment.

The transmitter unit 100 comprises a primary coil L₁, which, via an inverter 110 comprising four semiconductor switches (designated S₁ to S₄), for example MOSFETs or bipolar transistors, can be connected to a supply voltage U_{V} or can be supplied with this supply voltage. In addition, a prefilter 120 comprising unspecified components and a compensation capacitance are connected between the inverter 110 and the primary coil L₁. The compensation capacitance is used for resonant actuation (actuation with the design frequency) as reactive power compensation.

With the applied supply voltage U_{V} and suitable actuation of the inverter, an alternating magnetic field can thus be generated by means of the coil L₁.

The receiver unit 200 comprises a secondary coil L₂, to which a first intermediate circuit capacitor C_{Z,1} is connected via a compensation capacitance and a rectifier 210. A second intermediate circuit capacitor C_{Z,2} is connected to the first intermediate circuit capacitor C_{Z,1} via a switch S₇. The two intermediate circuit capacitors can therefore be connected in parallel or separate from one another by means of the switch S₇. In the present example, the switch S₇ is designed as an N-MOSFET, with a drain connection D, a source connection S, and a gate/switching connection G.

In turn, a power unit or power stage 240 is connected to the second intermediate circuit capacitor C_{Z,2}, said power unit comprising two semiconductor switches S₅ and S₆, which can be configured e.g. as MOSFETs, IGBTs, or bipolar transistors and, together with an inductance and a capacitance, serve as a buck converter, in particular.

An energy storage unit 220 and a consumer 225 are then connected to the power unit 240, for example. The energy storage unit can be separated from the power unit 240, for example, using an unspecified switch 221, for example in the case of a fault. The energy storage unit 220 can be an accumulator or a rechargeable battery, in particular. Using the aforementioned buck converter, a voltage Uₒᵤₜ with a current Iₒᵤₜ can be set at the energy storage unit, for example.

The rectifier 210 is designed as a passive rectifier with four unspecified diodes. However, the use of an active rectifier with, for example, semiconductor switches is also conceivable.

The receiver unit 200 further comprises an auxiliary supply unit 250, which is connected to the rectifier 210, for example, by means of an unspecified switch, a diode, and a capacitor—inter alia—and functions as a buck converter similarly to the power unit 240. In this way, the auxiliary supply unit 250 can itself be directly supplied with the voltage induced in the secondary coil L₂ and generate a specified, regulated output voltage U_{out,H}. The output voltage U_{out,H} is regulated by means of so-called pulse width modulation, i.e. via the switch-on time of the active switch of the auxiliary supply unit 250 in relation to the switching period.

Further, a (conventional) voltage comparator 252 is provided, to which the output voltage U_{out,H} of the auxiliary supply unit 250 is applied and which is connected to an optical coupler 251. The optical coupler 251 is, in turn, connected to the gate connection G of the switch/N-MOSFET S7.

The voltage comparator 252 and the optical coupler 251 are configured to flip or close the switch S₇ conductively and thus connect the second intermediate circuit capacitor C_{Z,2} together with the power unit 240 to the first intermediate circuit capacitor C_{Z,1} when the output voltage U_{out,H} exceeds a threshold value that can be appropriately specified or set. The voltage comparator 252 is non-inverting and switches its output voltage when a positive threshold value (preferably approx. 15 V) is reached at the input.

The receiver unit 200 can now in particular be configured to be arranged or implanted under a skin, indicated here with the number 310, and used for a circulatory or cardiac support system, for example. In particular, the energy storage unit 220 can be used for the energy supply of such a circulatory or cardiac support system.

When the transmitter unit 100 is positioned correspondingly outside or on the skin 310, a coupling between the primary coil L₁ of the transmitter unit 100 and the secondary coil L₂ of the receiver unit 200 is achieved, when positioned accordingly.

If the transmitter unit is now actuated or operated in such a way that an alternating magnetic field is generated by means of the primary coil L₁, a voltage or current flow is induced in the secondary coil L₂ by the coupling. This in turn leads to the first intermediate circuit capacitor C_{Z,1} being charged and the auxiliary supply unit 250 being supplied with voltage.

As soon as the output voltage U_{out,H} of the auxiliary supply unit exceeds the threshold value, the power unit 240 is, as mentioned above, connected to the following components and also supplied with voltage. In this way, a safe powering-up of the receiver unit 200 can be achieved.

1.-10. (canceled)

11. A transcutaneous wireless energy transfer system comprising:
a transmitter unit comprising a primary coil configured to receive a supply voltage; and
a receiver unit comprising:
a secondary coil;
a power unit connected to an electrically powered device or an energy storage device;
a rectifier;
an auxiliary supply unit connected to the rectifier;
a first intermediate circuit capacitor connected to the secondary coil via the rectifier; and
a second intermediate circuit capacitor connected to the power unit, wherein the second intermediate circuit capacitor and the first intermediate circuit capacitor are connected via a switch,
wherein the auxiliary supply unit is configured to close the switch to connect the first intermediate circuit capacitor to the second intermediate circuit capacitor in response to an output voltage of the auxiliary supply unit exceeding a threshold value.

12. The transcutaneous wireless energy transfer system of claim 11, wherein the switch is a semiconductor switch.

13. The transcutaneous wireless energy transfer system of claim 11, wherein the switch is a metal-oxide-semiconductor field-effect transistor (MOSFET) or an insulated-gate bipolar transistor (IGBT).

14. The transcutaneous wireless energy transfer system of claim 11, wherein the auxiliary supply unit comprises an optical coupler configured to actuate the switch.

15. The transcutaneous wireless energy transfer system of claim 14, wherein the auxiliary supply unit further comprises a voltage comparator configured to actuate the optical coupler to close the switch in response to the output voltage of the auxiliary supply unit exceeding the threshold value.

16. The transcutaneous wireless energy transfer system of claim 11, wherein the auxiliary supply unit comprises a voltage divider configured to generate the output voltage.

17. The transcutaneous wireless energy transfer system of claim 11, wherein the power unit comprises a buck converter.

18. The transcutaneous wireless energy transfer system of claim 11, wherein the receiver unit is configured to be arranged underneath the skin of a human or an animal.

19. The transcutaneous wireless energy transfer system of claim 11, wherein the transmitter unit is configured to be arranged on the skin outside of a human or animal body

20. The transcutaneous wireless energy transfer system claim 11, wherein energy received by the receiver unit is configured to be used for circulatory or cardiac support systems.

21. A cardiac support system, comprising:
a transcutaneous wireless energy transfer system, the energy transfer system comprising:
a transmitter unit comprising a primary coil configured to receive a supply voltage; and
a receiver unit comprising:
a secondary coil;
a power unit connected to an energy storage device, the energy storage device configured to supply energy to the cardiac support system;
a rectifier;
an auxiliary supply unit connected to the rectifier;
a first intermediate circuit capacitor connected to the secondary coil via the rectifier; and
a second intermediate circuit capacitor connected to the power unit, wherein the second intermediate circuit capacitor and the first intermediate circuit capacitor are connected via a switch,
wherein the auxiliary supply unit is configured to close the switch to connect the first intermediate circuit capacitor to the second intermediate circuit capacitor in response to an output voltage of the auxiliary supply unit exceeding a threshold value.

22. The cardiac support system of claim 21, wherein the switch is a semiconductor switch.

23. The cardiac support system of claim 21, wherein the switch is a metal-oxide-semiconductor field-effect transistor (MOSFET) or an insulated-gate bipolar transistor (IGBT).

24. The cardiac support system of claim 21, wherein the auxiliary supply unit comprises an optical coupler configured to actuate the switch.

25. The cardiac support system of claim 24, wherein the auxiliary supply unit further comprises a voltage comparator configured to actuate the optical coupler to close the switch when the output voltage of the auxiliary supply unit exceeds the threshold value.

26. The cardiac support system of claim 21, wherein the auxiliary supply unit comprises a voltage divider configured to generate the output voltage.

27. The cardiac support system of claim 21, wherein the power unit comprises a buck converter.

28. The cardiac support system of claim 21, wherein the receiver unit is configured to be arranged underneath the skin of a human or animal.

29. The cardiac support system of claim 21, wherein the transmitter unit is configured to be arranged on the skin outside of a human or animal body
